(19) United States
(12) Patent Application Publication
(10) Pub. No.: US 2006/0289039 A1
(43) Pub. Date: Dec. 28, 2006

(54) Cleaning with liquid carbon dioxide

(75) Inventors: Jan Hamrefors, Taby (SE); Kenneth Lindqvist, Skarpnack (SE)

Correspondence Address: CROWELL & MORING LLP;INTELLECTUAL PROPERTY GROUP, P.O. BOX 14300, WASHINGTON, DC, 20044-4300, US

(73) Assignee: LINDE AG ZENTRALE PATENTABTEILUNG, HOELLRIEGELSKREUTH (DE)

(21) Appl. No.: 10/543,716

(22) PCT Filed: Jan. 15, 2004
(86) PCT No.: PCT/EP04/00267

§ 371 Date: Aug. 11, 2006

(30) Foreign Application Priority Data
Jan. 28, 2003 (DE) 03002026.7

(51) Int. Cl. B08B 7/00; A01H 5/02

(52) U.S. Cl. 134/33; 8/148; 8/147

(57) ABSTRACT

The invention relates to a method for cleaning objects in a cleaning vessel using liquid carbon dioxide, wherein the cleaning vessel is at least partly filled with liquid carbon dioxide. Prior to and/or during the cleaning operation the pressure within said cleaning vessel is raised to a value above the corresponding vapour pressure. A gas other than carbon dioxide is introduced into said cleaning vessel and at least a portion of the cleaning operation is performed after the introduction of said gas.

The invention relates to a method for cleaning objects in a cleaning vessel using liquid carbon dioxide, wherein the cleaning vessel is at least partly filled with liquid carbon dioxide, and wherein prior to and/or during the cleaning operation the pressure within said cleaning vessel is raised to a value above the corresponding vapour pressure.

Dry-cleaning using liquid carbon dioxide is known as an environmentally friendly cleaning technique with favourable cleaning properties which can be used to remove contaminants from garments or textiles as well as from metal, machinery, workpieces or other parts. It is further known that the cleaning performance of carbon dioxide dry-cleaning can be improved by subcooling the liquid carbon dioxide.

A method of this kind is for example known from U.S. Pat. No. 5,759,209. According to this US patent document a pressure vessel, which is loaded with the objects to be cleaned, is partly or completely filled with liquid carbon dioxide under pressure. The cleaning operation is performed at a temperature below the critical temperature and at a pressure below the critical pressure of carbon dioxide. For a portion of the cleaning the pressure is raised with the temperature of the liquid remaining constant in order to subcool the liquid carbon dioxide.

In addition, it is well-known that chemical solvents and detergents can increase the cleaning efficiency. Mechanical scouring agents can further be used to improve the cleaning process.

It is an object of the present invention to develop a cleaning method using liquid carbon dioxide with improved cleaning efficiency.

This object is achieved by a method for cleaning objects in a cleaning vessel using liquid carbon dioxide, wherein the cleaning vessel is at least partly filled with liquid carbon dioxide, and wherein prior to and/or during the cleaning operation the pressure within said cleaning vessel is raised to a value above the corresponding vapour pressure. A gas other than carbon dioxide is introduced into said cleaning vessel and at least a portion of the cleaning operation is performed after the introduction of said gas.

According to the invention the pressure within said cleaning vessel is increased to a value above the corresponding vapour pressure prior to and/or during the cleaning operation. In other words, during at least a portion of the cleaning operation the temperature of the liquid carbon dioxide is below the equilibrium temperature of the pressure of the gas phase, that is the cleaning is carried out under a kind of subcooled conditions.

The invention is based on the discovery that the cleaning performance can be increased by raising the pressure in the cleaning vessel above the corresponding vapour pressure of the liquid carbon dioxide. In the following that process of increasing the pressure above the corresponding vapour pressure of the liquid carbon dioxide will be referred to as subcooling the liquid carbon dioxide. Due to that subcooling the amount of gas bubbles in the liquid decreases and thus any additives or detergents in the liquid carbon dioxide can better penetrate the parts or garments to be cleaned.

Further the addition of another gas rather than carbon dioxide changes the density of the gas phase. By adding a gas with a lower density than carbon dioxide gas the density of the gas phase is lowered which increases the difference between the density of the liquid phase and the gas phase. That difference in density is directly related to the interaction between the liquid carbon dioxide and the parts to be cleaned when there is any kind of mechanical agitation in the cleaning vessel. For example when using a rotating drum to agitate the objects, the objects are at least partly circulated between the liquid and the gaseous phase. The rotating drum causes the objects to move into the gaseous phase and then to fall back into the liquid carbon dioxide, whereby producing a mechanical impact on the objects. That mechanical agitation is more or less proportional to the difference in density between the liquid and the gaseous phase.

The method to subcool the liquid carbon dioxide by adding a gas having a lower density than carbon dioxide gas has thus two positive effects: First, the chemical interaction between the objects to be cleaned, the liquid carbon dioxide and possible detergents is essentially improved due to the reduced number of gas bubbles in the liquid. Second, the mechanical agitation is improved due to the increased difference in density between the gaseous and the liquid phase

Preferably hydrogen or a noble gas, for example helium or argon, is introduced in said cleaning vessel. It has been found that the addition of such a gas to the carbon dioxide clearly improves the cleaning performance. In particular the use of helium has shown good cleaning results. The mixture of gaseous carbon dioxide and helium forms a homogeneous blend with high cleaning performance.

It is advantageous to add such an amount of that gas, for example helium, into the cleaning vessel that the pressure within the cleaning vessel is raised by 1 to 10 bars, preferably by 1 to 5 bars. The relation between helium gas and carbon dioxide gas should be in the range of 1/50 to 1/5 to achieve the best cleaning results.

Preferably in the case of cleaning garments the pressure within the cleaning vessel is raised by 2 to 10 bars above the equilibrium pressure, more preferably by 4 to 7 bars. In cleaning parts or workpieces it might be necessary to increase the pressure difference much more in order to reach better cleaning efficiency. This is in particular the case if for example ultrasonic cleaning is also used in the cleaning process. In such applications the pressure within the cleaning vessel may be raised up to more than 100 bars.

This “subcooling” can also be achieved by cooling the liquid phase instead of raising the pressure. Related to the temperature of the liquid it is preferred to subcool the liquid by about 1 to 30 K. These conditions have proven to be the optimum between the additional expenditure for the “subcooling” and the increase in cleaning efficiency.

To cool the liquid carbon dioxide it is advantageous to use a a cooling machine or to decrease the temperature of the liquid carbon dioxide by indirect heat exchange with a coolant, for example by indirect heat exchange with liquid nitrogen. Further it is preferable to cool the liquid carbon dioxide by the addition of liquid carbon dioxide having a lower temperature.

To achieve faster cleaning cycles the liquid carbon dioxide is cooled and, at the same time, the pressure of the gas phase is raised. By simultaneously decreasing the temperature of the liquid and increasing the gas temperature, the time will be reduced before the “subcooled state” is reached. The whole cleaning operation can be accelerated and the cleaning cycle can be shortened.

From an economical point of view it is preferred to pressurize the cleaning vessel with gaseous carbon dioxide after the introduction of said other gas into said cleaning vessel. That is, first the cleaning vessel is at least partly filled with liquid carbon dioxide, then the inventive addition of a gas other than carbon dioxide is carried out and finally the cleaning vessel is fully pressurized with carbon dioxide gas. Standard gas storage devices, like gas cylinders, can be used as the source for the gas. The inventive method can be carried out more economically and a faster cleaning cycle is achieved.

Pressurization means that the pressure is increased to the working pressure of the cleaning machine which is preferably above 50 bars but below the critical pressure of carbon dioxide; As already described, at least a portion of the cleaning is carried out at a pressure above the corresponding vapour pressure of the liquid carbon dioxide. The pressure can be raised prior to starting the cleaning operation or during the cleaning process.

In the practical application of carbon dioxide dry-cleaning the carbon dioxide is recovered after the cleaning process has been completed. For that reason the “dirty” cleaning fluid loaden with the contaminants is drained from the cleaning vessel and conducted to a recovery system.

Surprisingly, it has been found that the inventive mixture of carbon dioxide and the added gas makes a homogeneous blend that can be recovered through the existing recovery system which is normally used to recover the carbon dioxide only. The cleaning fluid comprising the added gas is removed from the cleaning vessel, fed to the existing recovery unit where the contaminants and impurities are unloaded and conducted to a high pressure storage tank. The recovered cleaning fluid, that is a mixture of carbon dioxide and the added gas, can be used in the next cleaning cycle. In that way the gas losses are minimized. In tests using helium as the additional gas it has been found that only 4 to 10% of the added helium gas will be lossed during each cleaning cycle. Thus the method is economically very viable.

It is advantageous to add detergents, surfactants, enzymes or other additives to the liquid carbon dioxide. Due to the subcooling boiling of the liquid carbon dioxide and thus the number of gas bubbles in the liquid is essentially reduced. Consequently the contact between the liquid, the additives and the parts to be cleaned is intensified.

In addition, the articles to be cleaned may be agitated in order to improve the cleaning performance. Preferably the agitation is carried out by putting the objects into a rotating drum located within the cleaning vessel.

Preferably, the whole cleaning operation is performed after the inventive addition of the other gas into the cleaning vessel. That is, first the other gas is introduced into the cleaning vessel and then the cleaning operation is started.

For some applications it might also be advantageous to add that gas only for a portion of the cleaning cycle. In that case a part of the cleaning operation is carried out with the inventive addition of the other gas, another part is performed in the conventional way.

The invention has proven to be particular advantageous in cleaning garments and textiles. Further, a number of tests showed that the inventive method is also suitable for cleaning metal parts, such as metal surfaces or electronic parts. In particular organic residues, such as oils and greases, can be easily removed using subcooled liquid carbon dioxide.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. Method for cleaning objects in a cleaning vessel using liquid carbon dioxide, comprising:
partly filling the cleaning vessel with liquid carbon dioxide
introducing a gas other than carbon dioxide into said cleaning vessel, and
performing at least a portion of a cleaning operation after the introduction of said gas,
wherein the pressure within said cleaning vessel is raised to a value above the corresponding vapour pressure and below the critical pressure of carbon dioxide prior to and/or during the cleaning operation.

13. Method according to claim 12, wherein said gas is hydrogen or a noble gas.

14. Method according to claim 13, wherein said gas is helium.

15. Method according to claim 12, wherein the pressure within the cleaning vessel is raised 1 to 10 bars by the introduction of said gas.

16. Method according to claim 15, wherein said gas is hydrogen or a noble gas.

17. Method according to claim 15, wherein said gas is helium.

18. Method according to claim 12, wherein the pressure within the cleaning vessel is raised by 4 to 7 bars by the introduction of said gas.

19. Method according to claim 18, wherein said gas is hydrogen or a noble gas.

20. Method according to claim 19, wherein said gas is helium.

21. Method according to claim 12, wherein said cleaning vessel is pressurized with gaseous carbon dioxide after the introduction of said gas into said cleaning vessel.

22. Method according to claim 21, wherein said gas is hydrogen or a noble gas.

23. Method according to claim 20, wherein said cleaning vessel is pressurized with gaseous carbon dioxide after the introduction of said gas into said cleaning vessel.

24. Method according to claim 15, wherein said cleaning vessel is pressurized with gaseous carbon dioxide after the introduction of said gas into said cleaning vessel.

25. Method according to claim 17, wherein said cleaning vessel is pressurized with gaseous carbon dioxide after the introduction of said gas into said cleaning vessel.

26. Method according to claim 18, wherein said cleaning vessel is pressurized with gaseous carbon dioxide after the introduction of said gas into said cleaning vessel.

27. Method according to claim 12, wherein, prior to and/or during the cleaning operation said liquid carbon dioxide is cooled.

28. Method according to claim 27, wherein said liquid carbon dioxide is cooled by a cooling machine or by indirect heat exchange with a coolant.

29. Method according to claim 27, wherein said liquid carbon dioxide is cooled by the addition of colder carbon dioxide.

30. Method according to claim 12, wherein the mixture of said carbon dioxide and said gas within said cleaning vessel is partly recovered.

31. Method according to claim 13 wherein said objects or said liquid carbon dioxide are mechanically agitated within said cleaning vessel.

32. Method according to claim 12, wherein garments, textiles workpieces or parts are cleaned.

33. Method according to claim 12, wherein said gas has a density lower than carbon dioxide gas.
